(19) United States
(12) Patent Application Publication
(10) Pub. No.: US 2011/0166539 A1
(43) Pub. Date: Jul. 7, 2011

(54) Mounting for an Ostomy Pouch

(76) Inventor: Paul Andrew Eakin, (US)

(21) Appl. No.: 12/766,253

(22) Filed: Apr. 23, 2010

(30) Foreign Application Priority Data
Jan. 5, 2010 (GB) 1000057.8

(51) Int. Cl. A61F 5/448

(52) U.S. Cl. 604/339

(57) ABSTRACT

A mounting for an ostomy pouch which includes a mounting plate having a flexible body portion and a flange section and a flexible pad secured to the flexible body portion and at least a portion of an upper face of the flange section. The flexible body portion includes a raised central section and defines an internal opening for receiving a stoma. The flange section extends from an outer peripheral edge of the flexible body portion. The raised section of the flexible body portion and the upper face of the flange section extend in the same direction. The flexible pad is secured to the flexible body portion and at least a portion of the upper face of the flange section. The mounting plate is attachable to at least one securing strap at a plurality of attachment locations. The flange section defines at least one aperture at each of the attachment locations.

### CROSS-REFERENCE TO RELATED APPLICATIONS

This application claims benefits from UK Patent Application No. 1000057.8, filed Jan. 5, 2010, the contents of which are hereby incorporated herein by reference.

### BACKGROUND OF THE INVENTION

1. Field of the Invention

The present invention relates to a mounting for an ostomy pouch, and in particular a mounting for enabling an ostomy pouch to be attached to an ostomate using a strap.

2. State of the Art

An ostomy pouch is a medical prosthetic that provides a means for collecting waste diverted from a biological system of a patient's body such as the colon, ileum, or urinary system.

An ostomy pouch assembly typically consists of a collection pouch and a mounting for attaching the collection pouch to a patient's, or ‘ostomate's’, body. U.S. Pat. No. 6,790,200 describes an ostomy pouch assembly having a mounting which includes a foam disc having a first surface comprising a hydrocolloid skin barrier plate for contacting and adhering to the ostomate's skin, and an opposing second surface which is connected to a mounting plate, which in turn connects to the collection pouch. The mounting plate is a flexible plastic disc with an annular flange for adhering to the foam disc and the collection pouch on opposing sides. The mounting plate further includes attachment loops which extend from the flange and allow attachment of a belt or strap to enable the pouch assembly to be mounted to the ostomate.

It has been found that attachment loops are prone to failure due to the repeated mechanical loading resulting from motion of the ostomate's body, and the weight of the collection pouch. Such failure can cause injury to the ostomate, as well as causing release of the pouch contents and generally providing an inconvenience to the ostomate.

### SUMMARY OF THE INVENTION

According to the present invention there is provided a mount for an ostomy pouch as described in the accompanying claims. In addition, there is provided an ostomy pouch assembly as described in the accompanying claims.

In an embodiment of the invention there is provided a mounting for an ostomy pouch comprising a mounting plate, a flexible pad and a plurality of attachment points. The mounting plate comprises a flexible body portion including a raised central section having an internal opening for receiving a stoma, and a flange section extending from the outer peripheral edge of the body portion and having an upper face extending in the same direction as the raised section. The flexible pad is secured to the body section and at least a portion of the upper face of the flange section. The plurality of attachment points are provided for connecting the mounting plate to one or more securing straps, and comprise apertures which are formed within the flange section. Forming the attachment apertures within the flange section provides them with significantly greater strength, as they are surrounded by a greater thickness of material. In addition, the stress exerted on the material surrounding the apertures is more evenly distributed through the flange, rather than being concentrated through the relatively thin arms of an attachment loop as in the prior art. It has been found that this arrangement typically provides a 25% increase in strength compared to the attachment loop arrangement of the prior art.

Forming the attachment apertures within the flange section also provides the advantage of reducing material waste and providing a simplified more robust structure to the mounting plate which is easier to manufacture.

The raised central section is preferably a truncated convex section. The body portion and flange section are preferably integrally formed.

The body portion is a substantially circular disc and the flange section comprises at least 4 straight edges oriented tangentially to the peripheral edge of the body section, which define a pair of substantially triangular flange portions on opposing sides of the body section in which the attachment apertures are formed. This provides the mounting plate with a substantially straight edged peripheral edge, which is simple to form during manufacture and also enables more evenly distributed stress lines through the flange.

The flange section has a substantially square or diamond shape, with two opposing corners defining the triangular portions within which the attachment apertures are formed and the other two corners being truncated.

The flexible pad may be adhered to the body section and to an annular portion of the upper face of the flange section adjacent the outer peripheral edge of the body section such that the portions of the flange section immediately surrounding the attachment apertures are not directly adhered to the flexible pad. This allows securement of the flexible pad to the mounting plate while enabling a securing strap or straps to be passed through the apertures.

The flexible pad is preferably a foam disc, and may comprise multiple layers including a main disc section, an intermediate disc section located intermediate the main disc section and the mounting plate, and a hypercolloidal skin barrier layer. The hypercolloidal skin barrier layer may include a self adhesive surface to enable the mounting to be adhered to the ostomate's skin. The mounting plate may formed from a flexible plastic, which enables it to be molded or otherwise conveniently formed.

The present invention will now be described by way of example only with reference to the following illustrative figures.

### BRIEF DESCRIPTION OF THE DRAWINGS

FIG. 1 shows a mounting of the prior art;

FIG. 2 shows an ostomy pouch mounting according to an embodiment of the invention;

FIG. 3 shows a top view of the mounting plate of the ostomy pouch mounting of FIG. 2.

### DETAILED DESCRIPTION OF THE PREFERRED EMBODIMENTS

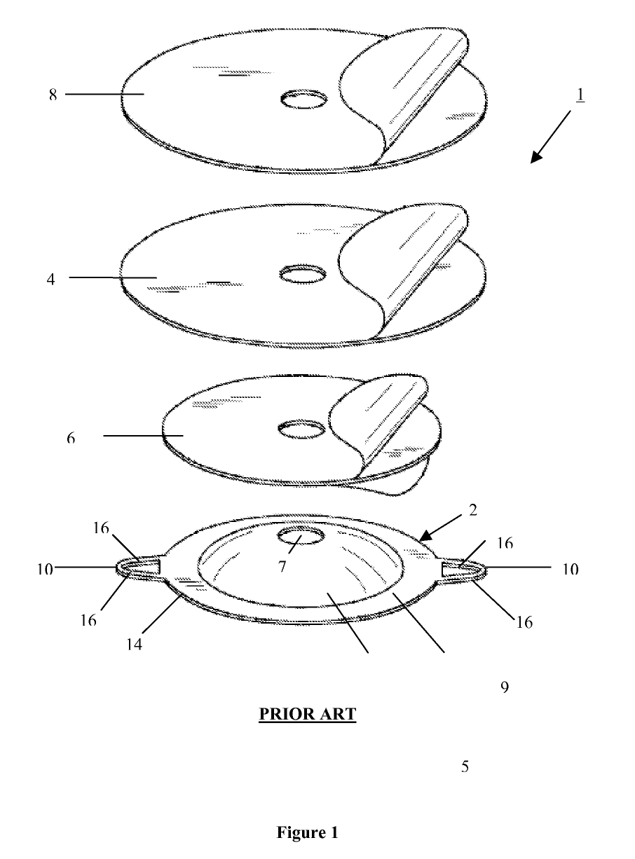

Referring to FIG. 1, the prior art mounting 1 includes a mounting plate 2. The mounting also includes a main foam disc section 4, an intermediate foam disc 6 and a hydrocolloid skin barrier layer 8 which are adhered together to form a flexible pad. The mounting disc 2 includes a central convex section 5 having an opening 7 defining a stoma portal, and an annular flange 9 extending from the central convex section 7. Strap loops 10 comprise arms 16 which extend from peripheral edge 14 of the annular flange 9. It has been found that strap loops 10 of this type are prone to failure under load, causing injury to the ostomate and/or release of the pouch contents as well as generally providing an inconvenience to the ostomate.

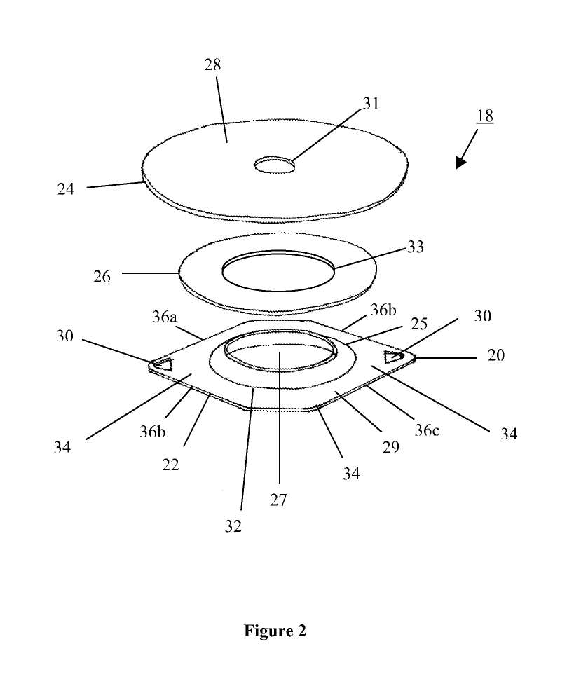

As shown in FIG. 2, the present invention provides a mounting 18 for mounting an ostomy pouch to an ostomate. The mounting 18 comprises a mounting plate 20, and a flexible pad comprising a hydrocolloid skin layer 28 and main foam disc 24 having an aperture 31 formed therein, and an intermediate foam disc 26 having an aperture 33.

The mounting plate 28 is formed from a flexible plastic material and comprises a central raised portion 25 in the form of a convex truncated hemisphere. An opening 27 is formed within the raised central portion 25 to define a stoma portal for receiving a stoma from the ostomate. A flange section 29 extends from the peripheral edge 32 of the central section 25. The aperture 33 is substantially equal in diameter to the aperture 27, which are both greater than the aperture 31. This enables the aperture 31 of the hydrocolloid layer 28 to be selectively cut to an appropriate size suitable for the associated stoma, up to a maximum diameter of the aperture 27.

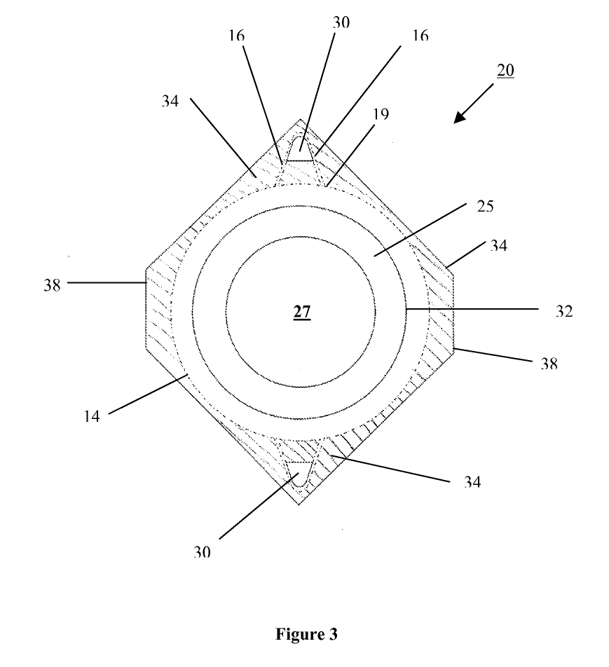

The flange section 29 is a planar element, and comprises a peripheral edge 22 including a series of straight edges 36 a-d which are spaced from and arranged tangentially to the peripheral edge 32 of the central section 25, and extend to define substantially triangular sections 34 on opposing sides of the central section 25. As can be seen from FIG. 3, the flange section 29 is substantially square in shape, with the triangular sections 34 arranged at opposing corners of the square, and the other corners 38 being truncated.

Attachment apertures 30 are formed in the triangular section 34 of the flange 29. These may be formed during molding, or subsequently stamp cut. The apertures 30 are configured to receive a belt or strap for mounting an ostomy collection pouch to an ostomate. In FIG. 3 the prior art mounting 2 of FIG. 1 is superimposed and shown in dashed lines. As can be seen, a significantly greater amount of material of the flange 29 surround the attachment apertures 30, as indicated by the hatched area, compared to the relatively thin arms 16 of the mounting 1. As such, the force exerted on the apertures 30 by the mounting strap in use is distributed through the flange 29 surrounding the apertures 30, thereby providing significantly greater strength. In contrast, rather than being formed within the confines of the flange 9, the thin arms 16 of the mounting 1 are connect to the flange 9 at connection points 19. As such, both the arms 16 and the connection points 19 provide potential failure points.

The flexible pad 24 is adhered to the upper surface of the central section 25 and to an annular portion of the upper surface 40 of the flange 29, and an ostomy collection pouch (not shown) is adhered to an annular portion of the opposing side of the mounting plate 20. Neither of the annular portions extends as far as the apertures 30. This ensures that the areas surrounding the apertures 30 are not adhered to the flexible pad 24 allowing access to the apertures 30 from both sides for the insertion of a belt or strap (not shown).

It will be appreciated that in further embodiments various modifications to the specific arrangements described above and shown in the drawings may be made, without departing from the scope of the invention as defined by the claims.

What is claimed is:

1. A mounting for an ostomy pouch, comprising:
a mounting plate comprising a flexible body portion and a flange section, said flexible body portion including a raised central section having an internal opening for receiving a stoma, said flange section extending from an outer peripheral edge of said flexible body portion and having an upper face, said raised section of said flexible body portion and said upper face of said flange section extending in the same direction; and
a flexible pad secured to the flexible body portion and at least a portion of the upper face of the flange section,
wherein said mounting plate is attachable to at least one securing strap at a plurality of attachment locations and the flange section defines at least one aperture at each of the attachment locations.

2. A mounting according to claim 1, wherein:
the flexible body portion and the flange section are integrally formed.

3. A mounting according to claim 1, wherein:
the body portion includes a substantially circular disc, the flange section comprises at least four straight edges oriented tangentially relative to the outer peripheral edge of the flexible body portion and defines a pair of substantially triangular flange portions on opposing sides of the flexible body portion, and the at least one aperture is defined in at least one of the pair of substantially triangular flange portions.

4. A mounting according to claim 3, wherein:
the flange section has a substantially square shape, two opposing corners which define the substantially triangular flange portions, and two additional corners which are truncated.

5. A mounting according to claim 1, wherein:
the upper face of the flange section defines an annular portion, and the flexible pad is adhered to the body portion and to the annular portion of the upper face adjacent the outer peripheral edge of the body portion such that portions of the flange section immediately surrounding the at least one aperture are not directly adhered to the flexible pad, and the securing strap is insertable through the apertures.

6. A mounting according to claim 1, wherein:
the mounting plate is formed from a flexible plastic.

7. A mounting according to claim 1, wherein:
the raised central section is a truncated convex section.

8. An ostomy pouch assembly, comprising:
an ostomy collection pouch; and
a mounting according to claim 1, the mounting including a mounting plate comprising a flexible body portion and a flange section, said flexible body portion including a raised central section having an internal opening for receiving a stoma, said flange section extending from an outer peripheral edge of said flexible body portion and having an upper face, said raised section of said flexible body portion and said upper face of said flange section extending in the same direction; and a flexible pad secured to the flexible body portion and at least a portion of the upper face of the flange section, wherein said mounting plate is attachable to at least one securing strap at a plurality of attachment locations and the flange section defines at least one aperture at each of the attachment locations, and
wherein the ostomy collection pouch is secured to the mounting plate on the opposing side of the mounting plate to the flexible pad.
